(19) United States
(12) Patent Application Publication
(10) Pub. No.: US 2006/0110355 A1
(43) Pub. Date: May 25, 2006

(54) Anhydrous cosmetic composition capable of forming an organogel

(75) Inventor: Xavier Blin, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/266,427

(22) Filed: Nov. 4, 2005

Related U.S. Application Data
(60) Provisional application No. 60/629,325, filed on Nov. 19, 2004.

(30) Foreign Application Priority Data
Nov. 5, 2004 (FR) 04 52539

(51) Int. Cl. A61K 8/55

(52) U.S. Cl. 424/70.23

(57) ABSTRACT

The present invention relates to an anhydrous cosmetic composition containing, in a physiologically acceptable medium, at least one fatty phase comprising at least one short hydrocarbon-based ester and at least one non-hydrogenated glycerophospholipid, the said short hydrocarbon-based ester and the said non-hydrogenated glycerophospholipid being present therein, respectively, in contents corresponding to those required in a binary mixture consisting of the said non-hydrogenated glycerophospholipid and short hydrocarbon-based ester to give it the ability to form an organogel by placing in contact with an aqueous medium.

The present invention relates to an anhydrous cosmetic care and/or makeup composition capable of forming an organogel on contact with an aqueous medium.

The cosmetic compositions targeted by the present invention are more particularly makeup and/or care products intended to be applied to keratin materials, especially the skin, the lips and/or the integuments, and may be formulated especially as lipsticks, lip balms, lip pencils, liquid or solid foundations, especially cast in stick or dish form, concealer products and skin-colouring products, temporary tattoos, eye makeup products, for instance eyeliners, in particular in pencil form, and mascaras, especially in cake form, or alternatively eyeshadows.

The staying power of a cosmetic composition, and especially the fastness of its colour, in particular in the case of a lipstick, is one of the determining characteristics in the production of an aesthetic effect. It is particularly difficult to obtain and forms the subject of considerable active research. It is in particular difficult to successfully combine within a cosmetic composition good staying power properties with satisfactory gloss. Moreover, besides its capacity to impart an aesthetic appearance, a cosmetic composition also needs to be pleasant to apply and to wear, and should not cause any tacky feel and/or greasy feel and/or sensation of drying-out.

In order to improve the staying power and hold of a homogeneous deposit of a cosmetic composition, it has especially been proposed to use volatile components in the formulation of these compositions. The evaporation of these components is reflected by concentration of the deposit of the composition, resulting in a reduction in the sensitivity to wear of this deposit. However, these compositions have the drawback of causing a sensation of drying-out.

Another alternative consists in structuring the deposit of the cosmetic composition in the form of a gel, in particular a hydrogel. Lipsticks have thus been developed comprising a combination of silicone surfactants capable of forming an aqueous gel by placing in contact with water (provided by the saliva), thus reflected by thickening of the deposit. However, the gel thus formed remains water-sensitive.

Other structures of gel type consisting of two immiscible phases (of water-in-oil type), which may contain hydrophilic active agents, have been described in patent U.S. Pat. No. 6,342,238. They are gelled and stabilized by means of a lecithin, which is generally hydrogenated.

Lecithin is a mixture of compounds containing phosphatidylcholine as predominant component, which is a molecule belonging to the glycerophospholipid family.

Lecithin is usually used in low proportion (in general less than 10%) as surfactant for the preparation of cosmetic compositions in the form of an aqueous or anhydrous emulsion, for example lipsticks (see especially WO 02/098378, WO 02/098379, WO 98/52927, US 2002/0 048 597 and EP 0 062 896).

Anhydrous compositions comprising a lipophilic phase and a polar solvent or a mixture of polar solvents have also been proposed, alone or in combination with other surfactants, for structuring purposes, in the form of inverse micelles and of lyotropic liquid crystal structures, as described in documents U.S. Pat. No. 6,325,995 and WO 95/11000. The polar solvents described in these documents are by nature hydrophilic solvents.

Documents EP 0 534 823 and WO 02/47617 use it to formulate anhydrous cosmetic compositions whose application to the skin or the lips is reflected by the spontaneous formation, in the presence of an aqueous medium, of lipid vesicles, or liposomes, promoting the release and activity of cosmetic and/or dermopharmaceutical active agents from these compositions.

However, these cosmetic compositions cannot combine improved fastness of the colour and satisfactory gloss. Moreover, their application, especially to the lips, is reflected by a tacky and greasy feel, or even also a sensation of drying-out.

The aim of the present invention is, specifically, to provide a care and/or makeup composition for keratin materials, such as the skin and/or the lips, that is directed towards satisfying all of the requirements mentioned above.

The inventors have thus discovered that it is possible to use at least one non-hydrogenated glycerophospholipid combined with a particular organic solvent to formulate an anhydrous cosmetic composition capable of forming an organogel by placing in contact with an aqueous medium.

Thus, according to one of its first aspects, one subject of the present invention is an anhydrous cosmetic composition containing, in a physiologically acceptable medium, at least one fatty phase comprising at least one short hydrocarbon-based ester and at least one non-hydrogenated glycerophospholipid, the short hydrocarbon-based ester and the non-hydrogenated glycerophospholipid being present therein, respectively, in contents corresponding to those required in a binary mixture consisting of the said non-hydrogenated glycerophospholipid and short ester, to give it the ability to form an organogel by placing in contact with an aqueous medium.

Cosmetic compositions comprising a binary mixture of this nature also have an ability to form an organogel by placing in contact with an aqueous medium.

For the purposes of the present invention, the expression “non-hydrogenated glycerophospholipid” is used to denote a glycerophospholipid comprising at least one fatty acid comprising at least one unsaturation.

For the purposes of the present invention, the term “required content” means the minimum content of a component that is required to obtain the expected effect, i.e., in the case of the non-hydrogenated glycerophospholipid and the short hydrocarbon-based ester, the production of an organogel by placing in contact with an aqueous medium.

For the purposes of the present invention, the term “aqueous medium” means a medium having a sufficient water content to convert into an organogel either the cosmetic composition according to the invention, applied to a support to be made up, or a non-hydrogenated glycerophospholipid/short hydrocarbon-based ester binary mixture. This aqueous medium may be provided, for example, by sweat, saliva, tears or any other natural or artificial liquid, for instance water, a water-in-oil emulsion or a lotion, which may be placed on the support to be made up before or after applying a cosmetic composition in accordance with the invention.

The formation of the organogel, following contact of the composition with an aqueous medium, for instance saliva, sweat or water, on the support under consideration, advantageously leads to the formation of a deposit having improved staying power and satisfactory gloss. Moreover, this deposit is also reflected by a pleasant sensation of softness and comfort, with a substantially reduced, or even nonexistent, tacky feel and/or greasy feel.

According to another of its aspects, a subject of the present invention is an anhydrous cosmetic composition containing, in a physiologically acceptable medium, at least one fatty phase comprising at least one short hydrocarbon-based ester and at least one non-hydrogenated glycerophospholipid, the said composition being free of hydrophilic polar solvent.

For the purposes of the present invention, the term “hydrophilic polar solvent” is intended to denote a solvent that is soluble in water at room temperature (25° C.) and at a concentration of greater than 5% by volume, for example a hydrocarbon-based solvent comprising at least one alcohol function, especially ethanol, isopropanol, hexanol, propanol, benzyl alcohol, propylene glycol, polypropylene glycol, butylene glycol, maltitol, sorbitol or glycerol, and mixtures thereof.

According to another of its aspects, a subject of the present invention is also an anhydrous cosmetic composition containing, in a physiologically acceptable medium, at least one fatty phase comprising at least one short hydrocarbon-based ester and at least one non-hydrogenated glycerophospholipid, the said composition comprising less than 5% by weight of wax relative to the total weight of the composition.

The cosmetic compositions according to the invention may be in the form of paste, gel, liquid, cream, semi-solid or solid. In particular, the cosmetic compositions according to the invention are in cast form, and more particularly in the form of a stick.

For the purposes of the present invention, the term “composition in cast form” is intended to denote a solid or semi-solid composition obtained after cooling a composition introduced in molten form into a mould. The compositions may be cast in the form of a stick or a wand, or in a dish.

According to another aspect, a subject of the present invention is also an anhydrous lipstick containing, in a physiologically acceptable medium, at least one fatty phase comprising at least one short hydrocarbon-based ester and at least one non-hydrogenated glycerophospholipid, the said lipstick being free of hydrophilic polar solvent.

According to yet another aspect, a subject of the present invention is also an anhydrous lipstick containing, in a physiologically acceptable medium, at least one fatty phase comprising at least one short hydrocarbon-based ester and at least one non-hydrogenated glycerophospholipid, the said lipstick comprising less than 5% by weight of wax relative to the total weight of the composition.

According to another of its aspects, a subject of the present invention is also a cosmetic process for caring for, making up and/or treating keratin materials, comprising at least the step of applying to these keratin materials a cosmetic composition in accordance with the present invention.

For the purposes of the present invention, the term “keratin material” is intended to denote any part of the human (or animal) body containing at least one layer of keratin, and especially the skin, the lips, the hair, the nails, the eyebrows and the eyelashes.

According to another aspect, the present invention also relates to a made-up synthetic support comprising, on all or part of its surface, at least one coat of a composition in accordance with the present invention.

According to yet another of its aspects, a subject of the present invention is also the use of at least one non-hydrogenated glycerophospholipid in combination with at least one short ester to give a cosmetic composition the ability to form an organogel by placing in contact with an aqueous medium.

According to yet another of its aspects, a subject of the present invention is also a care and/or makeup kit comprising:
at least a first composition comprising water, and
at least a second anhydrous composition containing, in a physiologically acceptable medium, at least one fatty phase comprising at least one short hydrocarbon-based ester and at least one non-hydrogenated glycerophospholipid.

The first composition comprises water in an amount sufficient for the second composition to form an organogel when it is placed in contact with the first composition.

The first composition may optionally comprise fragrances, dyes and/or active agents, especially cosmetic and/or dermatological active agents. The first composition may be applied to a keratin material, for example using a sprayer, an aerosol, a pump-dispenser bottle, by hand or using a handkerchief or a sponge.

The cosmetic compositions in accordance with the present invention may have improved staying power, and especially improved colour fastness.

In general, the compositions in accordance with the present invention may also have reduced transfer onto fabrics and reduced migration in wrinkles and fine lines.

Similarly, the cosmetic compositions in accordance with the present invention may also generally have good properties of hold of the deposit over time, while at the same time maintaining satisfactory gloss and comfort properties.

Moreover, the compositions in accordance with the present invention may also generally have a substantially reduced, or even nonexistent, greasy feel and tacky feel.

### NON-HYDROGENATED GLYCEROPHOSPHOLIPID

For the purposes of the present invention, the term “non-hydrogenated glycerophospholipid” means an ester obtained by reacting glycerol with at least one unsaturated fatty acid and phosphoric acid, the said phosphoric acid being substituted with a compound chosen from alcohols bearing an amine function, especially a β-amino alcohol. The β-amino alcohol may be chosen, for example, from choline, ethanolamine and serine.

The non-hydrogenated glycerophospholipid may be defined especially according to the general formula (I) below: 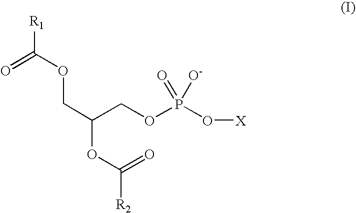
in which:

R₁ and R₂ represent, independently of each other, a saturated or unsaturated fatty acid containing from 4 to 24 carbon atoms, which may be branched, and which may be substituted with one or more hydroxyl and/or amine functions, and

X represents a substituent of general formula R₃R₄R₅N⁺-CH(R₆)-CH₂— in which R₃, R_{4,} R₅ and R₆ represent, independently of each other, a hydrogen, alkyl groups containing from 1 to 6 carbon atoms and/or a carboxyl function. X may be chosen especially from choline, serine and ethanolamine.

The terms “unsaturated” and “unsaturation” are intended to denote the presence of at least one, or even more, double or triple bonds between two carbon atoms.

According to one embodiment, R₁ and R₂, independently of each other, are advantageously chosen from butyric acid, caproic acid, caprylic acid, capric acid, caproleic acid, lauric acid, lauroleic acid, myristic acid, myristoleic acid, palmitic acid, palmitoleic acid, stearic acid, oleic acid, linoleic acid, linolenic acid, arachidic acid, isostearic acid, dihydroxystearic acid and ricinoleic acid.

Thus, for the purposes of the present invention, the term “glycerophospholipid” is intended to denote a mixture of compounds of general formula (I) in which R₁ and R₂ represent, independently of each other, a fatty acid as defined above, and X represents a basic nitrogenous compound, also as defined above.

The non-hydrogenated glycerophospholipid that is suitable for the implementation of the invention is especially chosen from non-hydrogenated phosphatidylcholine, non-hydrogenated phosphatidylethanolamine and non-hydrogenated phosphatidylserine, and mixtures thereof.

According to one embodiment, the non-hydrogenated glycerophospholipid is advantageously an ester of glycerol, of unsaturated fatty acid, of phosphoric acid and of choline, also known as phosphatidylcholine (PC).

The non-hydrogenated glycerophospholipid that is suitable for implementation of the invention may be derived from a non-hydrogenated lecithin. The non-hydrogenated lecithin may especially comprise, predominantly, non-hydrogenated phosphatidylcholine as non-hydrogenated glycerophospholipid.

The non-hydrogenated phosphatidylcholine (PC) that is suitable for implementation of the compositions in accordance with the invention may be of “natural” or “synthetic” origin.

“Natural” PC may be obtained by extraction from animal or plant sources, for instance soybean, sunflower or eggs. The non-hydrogenated phosphatidylcholine obtained naturally, for instance from soybean, generally contains as glycerol-esterifying fatty acid palmitic acid, stearic acid, palmitoleic acid, oleic acid, linoleic acid, linolenic acid, and possibly C₂₀ to C₂₂ fatty acids.

“Natural” PC is advantageously used in non-hydrogenated form, i.e. the double bonds present on the fatty acids have not been reduced, or have been only partially reduced.

For the purposes of the present invention, the term “synthetic phosphatidylcholine” is intended to denote phosphatidylcholine comprising at least one fatty acid different from those that may be present in natural PCs.

The term “synthetic PC” is also intended to denote natural PC that has been subjected to modifications, such as partial hydrogenation, i.e. only a fraction of the double bonds present on the unsaturated fatty acids is maintained.

Among the sources of more or less purified non-hydrogenated phosphatidylcholine that are suitable for implementation of the cosmetic compositions in accordance with the present invention, mention may be made of Emulmetik 930 sold by the company Lucas Meyer.

According to one particular embodiment, the cosmetic compositions in accordance with the invention comprise a content of non-hydrogenated glycerophospholipid(s) of at least 5%, of at least 10%, in particular of at least 15% and more particularly of at least 20% by weight relative to the total weight of the composition.

The non-hydrogenated glycerophospholipid that is suitable for implementing the present invention may especially be introduced into the composition in the form of a non-hydrogenated lecithin. This is generally obtained by lipid extraction using apolar solvents, from plant or animal fats. This lipid fraction usually predominantly comprises glycerophospholipids, including phosphatidylcholine.

The animal or plant sources that may be used to extract non-hydrogenated lecithins are, for example, soybean, sunflower or eggs. The glycerophospholipids included, in high proportion, in these lecithins are mainly phosphatidylcholine and phosphatidyl-ethanolamine.

The non-hydrogenated lecithins that are suitable for implementation of the present invention may be lecithins derived from soybean, from sunflower or from egg and/or mixtures thereof.

The lecithins are usually provided in a form dissolved in fatty acids, triglycerides or other solvents, or in the form of powders or cakes.

They are usually mixtures of lecithins, the content of glycerophospholipids of which, in the products as marketed, generally ranges from about at least 15% to about at least 95%.

Advantageously, the non-hydrogenated lecithin used as starting material for the preparation of the composition according to the invention comprises at least 45% by weight, in particular at least 65% by weight, in particular at least 75% by weight, in particular at least 85% by weight and more particularly at least 95% by weight of non-hydrogenated glycerophospholipid relative to the total weight of the lecithin.

Among the non-hydrogenated lecithins that may be suitable for implementation of the cosmetic compositions in accordance with the present invention, mention may be made of the lecithins sold under the references Nattermann Phospholipid®, Phospholipon 80® and Phosale 75® by the company American Lecithin Company, and Epikuron 145V, Topcithin 300, Emulmetik 930 and Ovothin 200 sold by the company Lucas Meyer.

The content of non-hydrogenated lecithin in the cosmetic compositions in accordance with the present invention is adjusted by a person skilled in the art such that the content of non-hydrogenated glycerophospholipids is sufficient to give a non-hydrogenated glycerophospholipid/short hydrocarbon-based ester binary mixture the ability to form an organogel when it is placed in contact with an aqueous medium.

The non-hydrogenated lecithin may represent at least 5% by weight, for example at least 10% by weight, in particular at least 15% by weight and more particularly at least 20% by weight relative to the total weight of the composition.

### SHORT ESTERS

The cosmetic compositions comprise at least one short hydrocarbon-based ester.

For the purposes of the present invention, the term “short hydrocarbon-based ester” means a hydrocarbon-based ester containing less than 40 and more than 10 carbon atoms.

The short hydrocarbon-based esters may be non-hydroxylated monoesters or diesters and are more particularly monoesters, i.e. bearing only one ester function. These esters may be linear, branched or cyclic, and saturated or unsaturated. In particular, they are branched and saturated.

When they are unsaturated, they may advantageously comprise from 1 to 4 double bonds and in particular from 1 to 2 double bonds.

The hydrocarbon-based chains arranged on either side of the ester function in the monoester may also comprise one or more hydroxyl functions and/or one or more carboxyl functions and/or one or more amine functions.

The hydrocarbon-based ester is non-volatile. For the purposes of the present invention, the term “non-volatile oil” means an oil having a vapour pressure of less than 0.13 Pa.

In particular, the hydrocarbon-based esters that may be used in the cosmetic compositions in accordance with the present invention may correspond to the formula RCOOR′ in which RCOO represents a fatty acid residue containing from 2 to 28 carbon atoms, and R′ represents a hydrocarbon-based chain containing from 1 to 28 carbon atoms.

These esters are in particular non-volatile esters, especially of C₁₀ to C₂₅ and in particular of C₁₄ to C₂₂. They may be chosen from esters of C₂ to C₁₈ acids and, especially, of C₂ to C₂₀ alcohols or of C₂ to C₈ polyols or mixtures thereof, on condition that the ester contains at least 10 carbons.

Thus, the esters may be chosen from a non-limiting list comprising neopentanoic acid esters, for instance isodecyl neopentanoate, isotridecyl neopentanoate, isostearyl neopentanoate and octyldodecyl neopentanoate, isononanoic acid esters, for instance isononyl isononanoate, octyl isononanoate, isodecyl isononanoate, isotridecyl isononanoate and isostearyl isononanoate, but also isopropyl alcohol esters, such as isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, and propylene glycol 2-diethylhexanoate, and mixtures thereof.

The said ester may also be chosen from synthetic esters, especially of fatty acid, for instance purcellin oil, isopropyl myristate, ethyl palmitate or octyl stearate; hydroxylated esters, for instance isostearyl lactate, octyl hydroxystearate, diisopropyl adipate, and fatty alkyl heptanoates, octanoates or decanoates, and mixtures thereof.

Isononyl isononanoate, isostearyl neopentanoate and isopropyl palmitate, and mixtures thereof, are most particularly suitable for performing the invention.

The short hydrocarbon-based esters are advantageously used in a content that is sufficient to allow the dissolution of the non-hydrogenated glycerophospholipid.

This or these hydrocarbon-based ester(s) may be present in the cosmetic compositions in accordance with the present invention in a proportion ranging from 5% to 95%, in particular from 10% to 80% and more particularly from 20% to 60% by weight relative to the total weight of the composition.

### ORGANOGEL

The term “gel” means a soft solid or pseudo-solid system comprising at least two components, of which one, which is liquid, constitutes the majority.

For the purposes of the present invention, the term “organogel” means a system whose liquid component is an organic phase.

The organogels may be obtained by adding a small amount of water to a cosmetic composition according to the invention comprising at least one non-hydrogenated glycerophospholipid dissolved in at least one short hydrocarbon-based ester.

For the purposes of the present invention, the term “small amount of water” means the minimum amount of water required to obtain a gel from a solution of non-hydrogenated glycerophospholipid in a short hydrocarbon-based ester.

The short hydrocarbon-based ester/non-hydrogenated glycerophospholipid weight ratio required to obtain an organogel may be determined from a binary mixture of short hydrocarbon-based ester(s) and of non-hydrogenated glycerophospholipid(s) according to the following protocol.

Non-hydrogenated glycerophospholipid/short hydrocarbon-based ester binary mixtures are obtained by varying the content of glycerophospholipid relative to the amount of short ester. For example, the content of non-hydrogenated glycerophospholipid may range from 5% to 20%. The dissolution of the non-hydrogenated glycerophospholipid in the short ester is obtained for variable concentrations of short ester, especially by heating the mixture to a temperature of less than or equal to 70° C. After dissolution of the binary mixtures and cooling to room temperature (20-25° C.), water or an aqueous medium is gradually added with manual stirring at each mix, until a system appearing as homogeneous is obtained. For example, demineralized water may be used in the implementation of this protocol. The water is added in proportions ranging from 1% to 15% by weight relative to the total weight of the mixture.

The contents of short ester and of non-hydrogenated glycerophospholipid may be chosen especially such that the content of water to be added to the mixture to obtain the organogel does not exceed 15% by weight relative to the total weight of the initial mixture, and especially less than or equal to 10%, especially less than or equal to 8%, or even less than or equal to 7%, and better still less than or equal to 5%, or even less than or equal to 3%, or even less than or equal to 2.5%, or better still less than or equal to 2%, and more particularly less than or equal to 1.5% by weight relative to the total weight of the initial composition.

Similarly, in the composition according to the invention, the weight ratio between the short hydrocarbon-based ester and the non-hydrogenated glycerophospholipid(s) may range from 0.1 to 10, in particular from 0.2 to 5 or even from 0.5 to 3.

The weight ratio between the short hydrocarbon-based ester and the non-hydrogenated lecithin used as source of glycerophospholipid that is mixed to prepare the composition according to the invention may especially range from 0.1 to 10, especially from 0.2 to 5, or even from 0.5 to 3.

The formation of the organogel, once the cosmetic composition has been applied to the keratin materials, may be reflected by a substantial increase in viscosity. This increase in viscosity especially improves the staying power of the deposit.

The viscosity of the organogels that may be obtained from the binary mixtures described above may be determined at 20-25° C. using a rotary viscometer of Mettler RM 180 type. Advantageously, when a binary mixture suitable for implementing a cosmetic composition in accordance with the invention is in organogel form, it has a viscosity ranging from 30 to 50 poises.

The dynamic viscosity of the composition is measured with a Mettler RM 180 viscometer. The Mettler RM 180 machine (Rhéomat) may be equipped with different spindles depending on the order of magnitude of the viscosity that it is desired to measure. For a viscosity of between 0.18 and 4.02 Pa·s, the machine is equipped with a No. 3 spindle. For a viscosity of between 1 and 24 Pa·s, the machine is equipped with a No. 4 spindle, and for a viscosity of between 8 and 122 Pa·s, the machine is equipped with a No. 5 spindle. The viscosity is read on the machine in deviation units (DU). Reference is then made to charts provided with the measuring machine to obtain the corresponding value in poises.

The spin speed of the spindle is 200 rpm.

Once the spindle is in rotation, at a constant set spin speed (in the present case 200 rpm), the viscosity value of the composition may vary over time. Measurements are taken at regular time intervals until they become constant. The viscosity value that has become constant over time is the value retained as being the dynamic viscosity value of the composition according to the invention. Depending on the measuring system on which the machine is set up, the viscosity measurement is taken after 10 minutes.

According to one embodiment of the invention, a non-hydrogenated glycerophospholipid/short hydrocarbon-based ester binary mixture that is suitable for implementing a cosmetic composition according to the invention may especially have an ability to form an organogel having a viscosity ranging from 30 to 50 poises, in particular from 35 to 50 poises, more particularly from 40 to 50 poises and in particular from 45 to 50 poises.

### PHYSIOLOGICALLY ACCEPTABLE MEDIUM

The term “physiologically acceptable medium” denotes a non-toxic medium that may be applied to human skin, lips or keratin materials. The physiologically acceptable medium is generally suited to the nature of the support onto which the composition is to be applied, and also to the aspect in which the composition is intended to be packaged.

The cosmetic compositions in accordance with the invention are in anhydrous form, i.e. they do not comprise an amount of water sufficient to induce by itself the formation of an organogel from a mixture of non-hydrogenated glycerophospholipid and of short hydrocarbon-based ester, as defined above.

For the purposes of the present invention, the term “anhydrous” means a composition comprising less than 2.5% by weight of water, especially less than 2% by weight of water, or even less than 1% by weight of water, or even less than 0.5% by weight of water, or better still less than 0. 1% by weight of water relative to the total weight of the composition.

According to one embodiment variant, the composition according to the invention may be free of water.

It may especially be in the form of oily gels, oily liquids, pastes or sticks.

According to one embodiment, the physiologically acceptable medium that is suitable for implementing the invention is especially free of hydrophilic polar solvent. As hydrophilic polar solvents that may be excluded from the compositions according to the invention, mention may be made of water, alcohol such as ethanol, propyl alcohol, isopropyl alcohol, hexanol and benzyl alcohol; polyols such as propylene glycol, polypropylene glycol, butylene glycol, maltitol, sorbitol and glycerol; panthenol dissolved in glycerol; aromatic oils; and mixtures thereof.

Fatty Phase

The cosmetic compositions in accordance with the present invention contain a fatty phase comprising, besides at least one short hydrocarbon-based ester, especially oils and fatty substances that are solid at room temperature (20-25° C.) and atmospheric pressure.

The term “oil” means any fatty substance that is in liquid form at room temperature (20-25° C.) and at atmospheric pressure. The liquid fatty phase may also contain, besides oils, other compounds dissolved in the oils, such as gelling agents and/or structuring agents, as indicated below.

Although the short hydrocarbon-based esters defined above may correspond to the definition of the oils proposed herein, it is understood that when it is specified that the cosmetic compositions in accordance with the present invention may also contain an oil, this oil is different from a short hydrocarbon-based ester as defined above.

The oil(s), other than short hydrocarbon-based esters, may be present in a proportion of from 0.1% to 90% by weight, in particular from at least 1% to 80% by weight, more particularly from 5% to 60% by weight, especially from 10% to 40% by weight or even from 20% to 30% by weight relative to the total weight of the cosmetic composition according to the invention.

Among the oils, other than the short hydrocarbon-based esters, that are suitable for implementing the invention, oils in which the non-hydrogenated glycerophospholipid is soluble and those in which it is at least partially insoluble are distinguished.

The oils that are suitable for preparing the cosmetic compositions according to the invention may be silicone or non-silicone oils.

For the purposes of the present invention, the term “silicone oil” means an oil comprising at least one silicon atom, and especially at least one Si—O group.

The term “hydrocarbon-based oil” means an oil mainly containing hydrogen and carbon atoms and possibly oxygen, nitrogen, sulfur and/or phosphorus atoms.

Hydrocarbon-based oils that may be mentioned include:
hydrocarbon-based oils of animal origin,
hydrocarbon-based oils of plant origin such as phytostearyl esters, such as phytostearyl oleate, phytostearyl isostearate and lauroyl glutamate/octyldodecyl/-phytostearyl (Ajinomoto, Eldew PS203), triglycerides consisting of fatty acid esters of glycerol, the fatty acids of which may have varied chain lengths from C₄ to C₂₄, these chains possibly being linear or branched, and saturated or unsaturated; these oils are especially heptanoic or octanoic triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; shea butter; or caprylic/capric acid triglycerides, for instance those sold by the company Stéarineries duBois or those sold under the names Miglyol 810®, 812® and 818® by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as parleam, and squalane and mixtures thereof, and in particular hydrogenated polyisobutene,
polyol esters and pentaerythritol esters, for instance dipentaerythrityl tetrahydroxystearate/tetraisostearate,
esters of diol dimers and of diacid dimers such as Lusplan DD-DA5® and Lusplan DD-DA7®, sold by the company Nippon Fine Chemical and described in patent application FR 0 302 809 filed on 6 Mar. 2003, the content of which is incorporated into the present patent application by reference,
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,
higher fatty acids such as oleic acid, linoleic acid and linolenic acid, and mixtures thereof, and
dialkyl carbonates, the 2 alkyl chains possibly being identical or different, such as the dicaprylyl carbonate sold under the name Cetiol CC®, by Cognis.

The silicone oils that are suitable for implementing the compositions according to the invention may be polydimethylsiloxanes (PDMS), polydimethylsiloxanes comprising alkyl or alkoxy groups that are pendent and/or at the end of a silicone chain, these groups each containing from 2 to 24 carbon atoms, phenylsilicones, for instance phenyl trimethicones, phenyl dimethicones, phenyltrimethylsiloxydiphenylsiloxanes, diphenyl dimethicones, diphenylmethyldiphenyltrisiloxanes, and 2-phenylethyltrimethyl siloxysilicates, and mixtures thereof.

Among the oils described above, the use of at least one oil in which the non-hydrogenated glycerophospholipid is at least partially insoluble makes it possible to concentrate the glycerophospholipid in the hydrocarbon-based oily phase in which it is soluble, especially the short ester, and thus to promote the formation of the organogel.

According to one embodiment variant of the invention, the composition in accordance with the invention may also comprise at least one oil in which the non-hydrogenated glycerophospholipid is at least partially insoluble.

According to another embodiment variant of the invention, the oil in which the non-hydrogenated glycerophospholipid is at least partially insoluble is chosen from high molecular weight hydrocarbon-based oils, silicone oils and fluorohydrocarbon oils, and mixtures thereof.

According to another embodiment variant, the oil in which the non-hydrogenated glycerophospholipid is at least partially insoluble is, for example, a hydrocarbon-based oil of high molecular weight of polyisobutylene type.

According to another embodiment variant, the oil in which the non-hydrogenated glycerophospholipid is at least partially insoluble is, for example, a phenylsilicone oil, especially the polyphenyltrimethylsiloxydimethylsiloxane sold under the reference Belsim 1000® by the company Wacker.

The oil in which the non-hydrogenated glycerophospholipid is at least partially insoluble may be present in the cosmetic compositions according to the invention in a proportion ranging from 0 to 60% by weight, in particular from 5% to 40% by weight and more particularly from 10% to 25% by weight relative to the total weight of the composition.

According to one embodiment variant, the oil in which the non-hydrogenated glycerophospholipid is at least partially insoluble and the said glycerophospholipid is present in a weight ratio ranging from 0.5 to 3, especially from 1 to 2.5 or even from 1.5 to 2.

The liquid fatty phase of the cosmetic compositions according to the invention may be thickened, gelled or structured, where appropriate, by incorporating therein a fatty-phase-gelling agent, for instance fillers of silica type as defined below, with the proviso that the use of these components is compatible with the production of an organogel under the conditions required according to the invention.

Waxes and Pasty Compounds

The compositions according to the invention may also comprise at least one compound chosen from waxes and pasty compounds, and mixtures thereof.

According to one embodiment, the compositions according to the invention comprising a hydrophilic polar solvent contain less than 5% by weight of wax relative to the total weight of the composition. These compositions especially comprise less than 3% or even less than 1% by weight of wax relative to the total weight of the composition.

According to yet another variant, the compositions according to the invention containing a hydrophilic polar solvent are especially free of wax.

According to another embodiment, the compositions according to the invention free of hydrophilic polar solvent may comprise at least one wax, in a proportion especially of greater than 5%.

The wax is solid at room temperature (20-25° C.), with a reversible solid/liquid change of state, having a melting point of greater than 30° C., which may be up to 200° C., a hardness of greater than 0.5 MPa and having in the solid state an anisotropic crystal organization. It may be a hydrocarbon-based wax, a fluoro wax and/or a silicone wax and may be of animal, plant, mineral or synthetic origin. It may be chosen, for example, from beeswax, camauba wax, candelilla wax, paraffin waxes, hydrogenated castor oil, synthetic waxes, for instance polyethylene waxes (preferably with a molecular weight of between 400 and 600) or Fischer-Tropsch waxes, silicone waxes, for instance alkyl or alkoxy dimethicones containing from 16 to 45 carbon atoms, ceresins or ozokerites, for instance isoparaffins with a melting point of less than 40° C., such as EMW-0003 sold by the company Nippon Seirou, α-olefin oligomers, such as the polymers Performa V® 825, 103 and 260 sold by the company New Phase Technologies; ethylene-propylene copolymers, such as Performalene® EP 700, and microcrystalline waxes with a melting point of greater than 85° C., such as HI-MIC® 1070, 1080, 1090 and 3080 sold by Nippon Seirou, and mixtures thereof.

The wax used in the cosmetic compositions in accordance with the invention is advantageously, for example, a polyethylene wax. The wax may also be a C₂₀-C₆₀ microcrystalline wax, especially the wax Microwax HW sold by the company Paramelt.

According to one particular embodiment, the wax(es) used in the cosmetic compositions in accordance.with the invention may be present in a content ranging from 0 to 30%, especially from 5% to 20% or even from 8% to 17% by weight relative to the total weight of the composition.

The cosmetic compositions in accordance with the present invention may also comprise at least one pasty compound.

For the purposes of the present invention, the term “pasty compound” means a fatty compound with a reversible solid/liquid change of state, and comprising at a temperature of 23° C. a liquid fraction and a solid fraction. The term “pasty substance” also means polyvinyl laurate.

For the purposes of the invention, the pasty compound advantageously has a hardness at 20° C. ranging from 0.001 to 0.5 MPa and preferably from 0.002 to 0.4 MPa.

Among the pasty compounds that may be used in the composition according to the invention, mention may be made of lanolins and lanolin derivatives, for instance acetylated lanolins, oxypropylenated lanolins or isopropyl lanolate, and mixtures thereof. Esters of fatty acids or of fatty alcohols may also be used, especially those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be mentioned include hydrogenated castor oil derivatives, such as Thixinr® from Rheox, and hydrogenated cocoglycerides.

Advantageously, the pasty fatty substance may be a mixture of lauric/palmitic/cetylic/stearic acid triglycerides (50/20/10/10), especially the product sold by the company Jasol under the reference Softisan 100®.

Mention may also be made of polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) sold by the Japanese company Kokyu Alcohol Kogyo.

Mention may also be made of pasty silicone compounds such as high molecular weight polydimethylsiloxanes (PDMS) and in particular those containing pendent chains of the alkyl or alkoxy type containing from 8 to 24 carbon atoms, and having a melting point of 20-55° C., for instance stearyl dimethicones, especially those sold by the company Dow Corning under the trade names DC2503® and DC25514®, and mixtures thereof.

According to one embodiment variant, the pasty compound may be present in the cosmetic compositions according to the invention in a proportion ranging from 0 to 30% by weight and especially from 10% to 20% by weight relative to the total weight of the composition.

### ADDITIVES

The cosmetic compositions in accordance with the invention may also comprise any additive usually used in the field under consideration, with the proviso that these additives do not impair the ability of the compositions according to the invention to form an organogel by placing in contact with an aqueous phase.

The additives that may be suitable for implementing the invention may be chosen especially from dyestuffs, for instance nacres and pigments, fillers, antioxidants, film-forming agents and, where appropriate, film-forming auxiliaries, gelling agents, essential oils, preserving agents, fragrances, moisturizers, antiseptic agents, vitamins such as vitamin B3 and E, and derivatives thereof, UV-protecting agents, neutralizers and surfactants, and mixtures thereof.

Needless to say, a person skilled in the art will also take care to select the optional additional additives and/or the amount thereof such that, besides the ability to form an organogel, the advantageous properties of the composition according to the invention, i.e. staying power, gloss, non-tacky aspect, coverage and migration resistance especially, are not, or are not substantially, adversely affected by the envisaged addition.

Dyestuffs

According to one embodiment, the composition according to the invention may also contain at least one organic or mineral dyestuff, especially of the pigment or nacre type.

According to another embodiment, the composition according to the invention may also contain at least one dyestuff chosen from lipophilic dyes, hydrophilic dyes, pigments, nacres and materials with a specific optical effect, and mixtures thereof.

This dyestuff may be present in a proportion of from 0.01% to 50% by weight relative to the total weight of the composition, in particular from 0.5% to 40%, more particularly from 5% to 25%, especially from 0.01% to 20%, in particular from 0.1% to 10% or even from 2% to 5% by weight relative to the total weight of the composition.

The term “pigments” should be understood as meaning white or coloured, mineral or organic particles that are insoluble in an aqueous solution, which are intended to colour and/or opacify the resulting film.

The pigments may be present in a proportion of from 0.01% to 20% by weight, especially from 0.01% to 5% by weight and in particular from 0.02% to 7% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC BALL® PC-LL-100 P, this pigment consisting of silica microspheres containing yellow iron oxide.

Among the organic pigments that may be used in the invention, mention may be made of carbon black, pigments of D & C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica®, Flamenco® and Duochrome® (on mica base) sold by the company Engelhard, the Timiron® nacres sold by the company Merck, the Prestige® nacres on mica base sold by the company Eckart and the Sunshine® nacres on synthetic mica base sold by the company Sun Chemical.

The nacres may more particularly have a yellow, pink, red, bronze, orange, brown and/or coppery colour or glint.

The pigments may or may not be surface-coated, in particular surface-treated with silicones, amino acids, fluoro derivatives or any other substance that promotes the dispersion and compatibility of the pigment in the composition.

Advantageously, the pigments used in the compositions in accordance with the invention may also be surface-coated with a lecithin coating. This coating may be obtained by placing a solution of pigment in contact with a solution of lecithin, in the presence of divalent or trivalent metal salts. To obtain this coating, hydrogenated or non-hydrogenated lecithin may be used.

The cosmetic composition according to the invention may also comprise water-soluble or liposoluble dyes in a content ranging from 0.01% to 10% by weight and especially ranging from 0.01% to 5% by weight relative to the total weight of the cosmetic composition.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow.

When the cosmetic compositions according to the invention comprise a water-soluble dye, this dye may be present in the composition in dispersed form.

The cosmetic composition according to the invention may also contain at least one material with a specific optical effect.

This effect is different from a simple conventional hue effect, i.e. a unified and stabilized effect as produced by standard dyestuffs, for instance monochromatic pigments. For the purposes of the invention, the term “stabilized” means lacking the effect of variability of the colour with the angle of observation or in response to a temperature change.

For example, this material may be chosen from particles with a metallic glint, goniochromatic colouring agents, diffracting pigments, thermochromic agents, optical brighteners, and also fibres, especially interference fibres. Needless to say, these various materials may be combined so as to afford the simultaneous manifestation of two effects, or even of a novel effect in accordance with the invention.

The particles with a metallic glint that may be used in the invention are in particular chosen from:
particles of at least one metal and/or of at least one metal derivative,
particles comprising a monomaterial or multimaterial organic or mineral substrate, at least partially coated with at least one layer with a metallic glint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are preferred metals.

The term “metal derivatives” is intended to denote compounds derived from metals, especially oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe® 700 from the company Eckart, silica-coated aluminium particles sold under the name Visionaire Bright Silver® from the company Eckart, and metal alloy particles, for instance the silica-coated bronze (alloy of copper and zinc) powders sold under the name Visionaire Bright Natural Gold® from the company Eckart.

They may also be particles comprising a glass substrate, for instance those sold by the company Nippon Sheet Glass under the name Microglass Metashine®.

The goniochromatic colouring agent may be chosen, for example, from interference multilayer structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Carribean Blue® by the company Merck. Mention may also be made of Infinite Colors® pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the product sold under the name Helicone® HC by the company Wacker.

Fillers

Advantageously, the cosmetic compositions in accordance with the invention may also comprise at least one filler, of organic or mineral nature.

The term “filler” should be understood as meaning colourless or white solid particles of any form, which are in an insoluble form and dispersed in the medium of the composition. Being of mineral or organic nature they make it possible to impart body or rigidity to the composition, and/or softness, a matt effect and uniformity to the makeup.

The fillers used in the compositions according to the present invention may be of lamellar, globular or spherical form, fibres or in any form intermediate between these defined forms.

The fillers according to the invention may or may not be surface-coated, and in particular they may be surface-treated with silicones, amino acids, fluoro derivatives or any other substance that promotes the dispersion and compatibility of the filler in the composition.

For the purposes of the present invention, the terms “mineral fillers” and “inorganic fillers” are used interchangeably.

Among the mineral fillers that may be used in the compositions according to the invention, mention may be made of talc, mica, silica, trimethyl siloxysilicate, kaolin, bentone, hydroxyapatite, boron nitride, hollow silica microspheres (Silica Beads® from Maprecos), glass or ceramic microcapsules, silica-based fillers, for instance Aerosil® or Aerosil® 300; Sunsphare® L-3 1 and Sunsphare® H-31 sold by Asahi Glass; Chemicelen® sold by Asahi Chemical; composites of silica and of titanium dioxide, for instance the TSG series sold by Nippon Sheet Glass, and mixtures thereof.

Among the organic fillers that may be used in the compositions according to the invention, mention may be made of polyamide powders (Nylon® Orgasol from Atochem), poly-β-alanine powder and polyethylene powder, polytetrafluoroethylene powders (Teflon®), lauroylysine, starch, tetrafluoroethylene polymer powders, hollow polymer microspheres such as Expancel® (Nobel Industrie), precipitated calcium carbonate, magnesium carbonate, magnesium hydrogencarbonate, metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate, Polypore® L 200 (Chemdal Corporation), silicone resin microbeads (for example Tospearl® from Toshiba), polyurethane powders, in particular powders of crosslinked polyurethane comprising a copolymer, the said copolymer comprising trimethylol hexyllactone. In particular, it may be a polymer of hexamethylene diisocyanate/trimethylol hexyllactone. Such particles are especially commercially available, for example, under the name Plastic Powder D-400® or Plastic Powder D-800® from the company Toshiki, and mixtures thereof.

The fillers may be present in the cosmetic compositions in accordance with the invention in a proportion of from 0.001% to 60%, preferably 0.5% to 35%, especially from 0.5% to 20% or even from 1% to 10% by weight relative to the total weight of the composition.

The filler may be, for example, a filler whose mean particle size is less than 100 μm, especially between 1 and 50 μm, for example between 4 and 20 μm.

A subject of the present invention is also a cosmetic process for caring for, making up and/or treating keratin materials, comprising at least the step of applying to these keratin materials a cosmetic composition as defined above.

The surface to be made up may be prepared, prior to the application of the cosmetic composition according to the invention, by applying a composition comprising an aqueous medium.

According to another embodiment variant, this aqueous phase may be added after applying the cosmetic composition to the surface to be made up.

The aqueous medium may be saliva, tears or sweat. It may also be mineral water, spring water, tap water, sea water, an aqueous solution, an aqueous-alcoholic solution, or even an emulsion, especially an oil-in-water emulsion. The aqueous medium may also contain at least one compound chosen from a dye, a fragrance, a cosmetic and/or dermatological active agent, and mixtures thereof. The aqueous medium may be applied, for example, by vaporization or wetting.

According to yet another embodiment variant, the composition in accordance with the invention may be supplied independently or in combination with an aqueous medium.

According to yet another embodiment variant, when the aqueous medium is supplied in combination with a composition in accordance with the invention, it may be packaged in a single-dose or multi-dose container.

Thus, a composition according to the invention may be supplied in the form of a care and/or makeup kit comprising at least a first composition comprising water, and at least a second anhydrous composition containing, in a physiologically acceptable medium, at least one fatty phase comprising at least one short hydrocarbon-based ester and at least one non-hydrogenated glycerophospholipid.

The first composition comprises water in an amount sufficient for the second composition to form an organogel when it is placed in contact with the first composition.

The first composition comprising water may be, for example, an aqueous medium as defined above.

According to yet another embodiment, a subject of the present invention is also a process for caring for and/or making up keratin materials, comprising at least one step of applying, one over the other, a first composition comprising water, and a second anhydrous composition containing, in a physiologically acceptable medium, at least one fatty phase comprising at least one short hydrocarbon-based ester and at least one non-hydrogenated glycerophospholipid.

In the above process, the first composition and the second composition may be applied in any order.

According to one embodiment variant, the non-hydrogenated glycerophospholipid is introduced into the composition in accordance with the invention in the form of a non-hydrogenated lecithin in a proportion of about 10% by weight relative to the total weight of the composition. This lecithin may be a lecithin containing at least 95% by weight of non-hydrogenated glycerophospholipid relative to the total weight of the lecithin. This non-hydrogenated glycerophospholipid may be non-hydrogenated phosphatidylcholine.

According to another variant, the cosmetic composition in accordance with the present invention comprises non-hydrogenated phosphatidylcholine as non-hydrogenated glycerophospholipid, and at least isononyl isononanoate as short ester.

According to another variant, the cosmetic composition according to the invention comprises non-hydrogenated phosphatidylcholine as non-hydrogenated glycerophospholipid and a microcrystalline wax as wax.

According to yet another variant, the cosmetic composition according to the present invention comprises non-hydrogenated phosphatidylcholine as non-hydrogenated glycerophospholipid and a mixture of lauric/palmitic/cetylic/stearic acid triglycerides (50/20/10/10) as pasty compound.

The cosmetic composition according to the invention may especially be in the form of a lip makeup product, in particular in the form of a lipstick or a lip balm.

The composition examples below are given as non-limiting illustrations of the invention.

### EXAMPLE 1

### Binary Mixture

Binary mixtures of 20% non-hydrogenated lecithin, comprising at least 95% by weight of non-hydrogenated glycerophospholipid relative to the total weight of the lecithin (Emulmetik 930), and of 80% short hydrocarbon-based ester were prepared by stirring and heating up to around 70° C., until the lecithin has dissolved.

After cooling to room temperature, water is added to the mixture, with stirring, in a proportion of 1% relative to the total weight of the binary mixture.

The appearance of the compositions is observed after dissolving the lecithin and 24 hours after addition of water. The results are summarized in Table (I) below.

The results show that a binary mixture of non-hydrogenated lecithin and of short hydrocarbon-based ester, in proportions of 20% and 80%, respectively, is capable of forming an organogel by addition of a small proportion of water.

| | | Aspect of the | |
|---|---|---|---|
| | Aspect of the | mixture 24 hours | |
| | binary mixture | after addition | |
| Short ester | after dissolution | of 1% water | Conclusion |
| Isopropyl | Clear yellow | Transparent thick | Organogel |
| myristate | liquid | yellow gel | |
| Isopropyl | Clear yellow | Transparent thick | Organogel |
| palmitate | liquid | yellow liquid | |
| Isononyl | Clear pale | Transparent thick | Organogel |
| isononanoate | yellow liquid | yellow liquid | |

### EXAMPLE II

### Lipstick

TABLE II

| Control composition | | Composition A | |
|---|---|---|---|
| % by weight | | % by weight | |
| Diisostearyl malate | 22.71 | Isononyl isononanoate | 18.92 |
| Phenyltrimethylsiloxy- | 18.17 | Phenyltrimethylsiloxy- | 15.69 |
| trisiloxane | | trisiloxane | |
| (viscosity: 20 cSt) | | (viscosity: 20 cSt) | |
| Polyphenyltrimethyl- | 27.26 | Polyphenyltrimethyl- | 23.53 |
| siloxydimethylsiloxane | | siloxydimethylsiloxane | |
| (viscosity: 1000 cSt) | | (viscosity: 1000 cSt) | |
| | — | Non-hydrogenated lecithin | 10.00 |
| | | (Emulmetik 930) | |
| Microcrystalline wax | 10.00 | Microcrystalline wax | 10.00 |
| C30/C45 | 2.50 | C30/C45 | 2.50 |
| α,ω-alkylated | | α,ω-alkylated | |
| polydimethylsiloxane | | polydimethylsiloxane | |
| Lauric/palmitic/cetylic/ | 10.00 | Lauric/palmitic/cetylic/ | 10.00 |
| stearic acid | | stearic acid | |
| triglyceride (50/20/10/10) | | triglyceride (50/20/10/10) | |
| Diisostearyl malate | 5.00 | Isononyl isononanoate | 5.00 |
| Eosin | 0.06 | Eosin | 0.06 |
| Calcium salt of | 0.26 | Calcium salt of | 0.26 |
| lithol B red | | lithol B red | |
| Black iron oxide | 0.09 | Black iron oxide | 0.09 |
| Brown iron oxide | 2.15 | Brown iron oxide | 2.15 |
| Mica-titanium oxide | 1.80 | Mica-titanium oxide | 1.80 |
| (67.5/32.5) | | (67.5/32.5) | |

Procedure

Two lipstick formulations are prepared, a control composition and a composition in accordance with the invention, composition A.

A first oily phase is prepared by mixing at elevated temperature (at about 70° C.) the oils, a fraction of the short ester and the non-hydrogenated lecithin (when they are present, i.e. in composition A).

A second oily phase is prepared by mixing another fraction of the short ester and the dyes.

The waxes and the pasty compounds are then added to the mixture.

The mixture thus obtained is then cast in a lipstick mould and left to cool until a solid composition is obtained.

The lipstick formulated on the basis of a composition in accordance with the present invention (composition A) has a markedly reduced tacky feel compared with that of the control composition, while at the same time having improved staying power and satisfactory gloss.

1. Anhydrous cosmetic composition containing, in a physiologically acceptable medium, at least one fatty phase comprising at least one short hydrocarbon-based ester and at least one non-hydrogenated glycerophospholipid, the said composition being free of hydrophilic polar solvent.

2. Anhydrous cosmetic composition containing, in a physiologically acceptable medium, at least one fatty phase comprising at least one short hydrocarbon-based ester and at least one non-hydrogenated glycerophospholipid, the said composition comprising less than 5% by weight of wax relative to the total weight of the composition.

3. Anhydrous cosmetic composition containing, in a physiologically acceptable medium, at least one fatty phase comprising at least one short hydrocarbon-based ester and at least one non-hydrogenated glycerophospholipid, the said short hydrocarbon-based ester and the said non-hydrogenated glycerophospholipid being present therein, respectively, in contents corresponding to those required in a binary mixture consisting of the said non-hydrogenated glycerophospholipid and short hydrocarbon-based ester to give it the ability to form an organogel by placing in contact with an aqueous medium.

4. Composition according to any one of the preceding claims, characterized in that the non-hydrogenated glycerophospholipid and the short hydrocarbon-based ester are present therein, respectively, in contents corresponding to those required in a binary mixture consisting of the said non-hydrogenated glycerophospholipid and short hydrocarbon-based ester to give it the ability to form an organogel having a viscosity ranging from 30 to 50 poises, in particular from 35 to 50 poises, more particularly from 40 to 50 poises and in particular from 45 to 50 poises.

5. Cosmetic composition according to any one of the preceding claims, characterized in that the short ester and the non-hydrogenated glycerophospholipid are present in a weight ratio ranging from 0.1 to 10, in particular from 0.2 to 5 or even from 0.5 to 3.

6. Composition according to any one of the preceding claims, characterized in that the said non-hydrogenated glycerophospholipid is present in a content of at least 5%, of at least 10%, in particular of at least 15% and more particularly of at least 20% by weight relative to the total weight of the composition.

7. Composition according to any one of the preceding claims, characterized in that the non-hydrogenated glycerophospholipid is chosen from non-hydrogenated phosphatidylcholine, non-hydrogenated phosphatidylethanolamine and non-hydrogenated phosphatidyl serine, and mixtures thereof.

8. Cosmetic composition according to any one of the preceding claims, characterized in that the non-hydrogenated glycerophospholipid is introduced into the said composition in the form of a non-hydrogenated lecithin.

9. Composition according to the preceding claim, characterized in that the non-hydrogenated lecithin is chosen from lecithins derived from soybean, from sunflower and from egg, and mixtures thereof.

10. Cosmetic composition according to claim 8 or 9, characterized in that the said non-hydrogenated lecithin comprises at least 45%, in particular at least 65% by weight, in particular at least 75% by weight, in particular at least 85% by weight and more particularly at least 95% by weight of non-hydrogenated glycerophospholipid relative to the total weight of the lecithin.

11. Composition according to any one of claims 8 to 10, characterized in that the non-hydrogenated lecithin is present in a proportion of at least 5%, of at least 10% by weight, in particular of at least 15% by weight and more particularly at least 20% by weight relative to the total weight of the composition.

12. Composition according to any one of the preceding claims, characterized in that the said short hydrocarbon-based ester is chosen from non-hydroxylated monoesters and diesters, the said monoesters and diesters containing less than 40 carbon atoms.

13. Composition according to any one of the preceding claims, characterized in that the said short hydrocarbon-based ester is chosen from isononyl isononanoate, isostearyl neopentanoate and isopropyl palmitate, and mixtures thereof.

14. Composition according to any one of the preceding claims, characterized in that the said short hydrocarbon-based ester is present in a proportion ranging from 5% to 95%, in particular from 10% to 80% and more particularly ranging from 20% to 60% by weight relative to the total weight of the composition.

15. Composition according to any one of the preceding claims, characterized in that it also comprises at least one oil in which the said non-hydrogenated glycerophospholipid is at least partially insoluble.

16. Composition according to the preceding claim, characterized in that the said oil is chosen from high molecular weight hydrocarbon-based oils, silicone oils and fluorohydrocarbon oils, and mixtures thereof.

17. Composition according to claim 15 or 16, characterized in that the said oil is present in a proportion ranging from 0 to 60% by weight, in particular from 5% to 40% by weight and more particularly from 10% to 25% by weight relative to the total weight of the composition.

18. Composition according to any one of claims 15 to 17, characterized in that the said oil and the said non-hydrogenated glycerophospholipid are present in a weight ratio ranging from 0.5 to 3, especially from 1 to 2.5 or even from 1.5 to 2.

19. Composition according to any one of claims 15 to 18, characterized in that the said oil is a phenylsilicone oil.

20. Composition according to any one of the preceding claims, characterized in that it also comprises at least one compound chosen from pasty fatty substances and waxes, and mixtures thereof.

21. Composition according to the preceding claim, characterized in that the said compound is a polyethylene wax.

22. Composition according to claim 20, characterized in that the said compound is a pasty substance chosen from lanolin and its derivatives, esters of fatty acids or of fatty alcohols, and high molecular weight polydimethylsiloxanes, and mixtures thereof.

23. Composition according to any one of the preceding claims, characterized in that it also comprises at least one additive chosen from fillers, pigments, antioxidants, essential oils, preserving agents, neutralizers, fragrances and surfactants, and mixtures thereof.

24. Composition according to any one of the preceding claims, characterized in that it is in the form of a lipstick.

25. Cosmetic process for caring for, making up and/or treating keratin materials, comprising at least the step of applying to these keratin materials a cosmetic composition in accordance with any one of the preceding claims.

26. Made-up synthetic support comprising, on all or part of its surface, at least one coat of a cosmetic composition in accordance with any one of the preceding claims.

27. Cosmetic use of at least one non-hydrogenated glycerophospholipid in combination with at least one short hydrocarbon-based ester to give a cosmetic composition the ability to form an organogel.

28. Use according to claim 27, characterized in that the said non-hydrogenated glycerophospholipid is as defined according to any one of claims 3 to 11.

29. Use according to claim 27 or 28, characterized in that the said short hydrocarbon-based ester is as defined according to any one of claims 12 to 14.

30. Care and/or makeup kit comprising:
at least a first composition comprising water, and
at least a second anhydrous composition containing, in a physiologically acceptable medium, at least one fatty phase comprising at least one short hydrocarbon-based ester and at least one non-hydrogenated glycerophospholipid.

31. Kit according to the preceding claim, characterized in that the first composition comprises water in an amount sufficient for the second composition to form an organogel when it is placed in contact with the first composition.

32. Kit according to claim 30 or 31, characterized in that the first composition is an aqueous solution or an oil-in-water emulsion.

33. Kit according to any one of claims 30 to 32, characterized in that the first composition comprises at least one cosmetic and/or dermatological active agent.

34. Kit according to either of claims 30 and 33, characterized in that the first composition is packaged in a single-dose or multi-dose container.

35. Process for making up keratin materials, comprising at least one step of applying, one over the other:
a first composition comprising water, and
a second anhydrous composition comprising, in a physiologically acceptable medium, at least one fatty phase comprising at least one short hydrocarbon-based ester and at least one non-hydrogenated glycerophospholipid.
